(19) United States
(12) Patent Application Publication
(10) Pub. No.: US 2025/0058309 A1
(43) Pub. Date: Feb. 20, 2025

(54) METHOD FOR CARRYING OUT A CHEMICAL REACTION IN AN UPFLOW REACTOR

(71) Applicant: SABIC GLOBAL TECHNOLOGIES B.V., Bergen op Zoom (NL)

(72) Inventors: Suman Kumar Sen, Bangalore (IN); Prashant Patil, Bangalore (IN); Surya Prakasa Rao Daliparthi, Bangalore (IN); Dwairath Dhar, Bangalore (IN); Amit Kumar Mondal, Hosur (IN); Paulus Johannes Maria Eijsbouts, Nieuwkuijk (NL); Rudolf Martinus Petrus van der Heijden, Geleen (NL); Gert-Jan Schoenmakers, Prinsenbeek (NL); Mouli Murukutla, Malur (IN)

(21) Appl. No.: 18/721,057

(22) PCT Filed: Dec. 12, 2022
(86) PCT No.: PCT/EP2022/085306

§ 371 Date: Jun. 17, 2024

(30) Foreign Application Priority Data
Dec. 17, 2021 (EP) 21215714.3
Dec. 17, 2021 (EP) 21215716.8

(51) Int. Cl. B01J 35/53; B01J 35/40; B01J 37/00; C07C 37/20

(52) U.S. Cl. CPC B01J 35/53; B01J 35/40; B01J 37/0072; C07C 37/20; B01J 2235/00; B01J 2523/62

(57) ABSTRACT

The present invention relates to a method for carrying out a catalysed chemical reaction using one or more liquid reactants, preferably acetone and phenol to form bisphenol A, in an upflow reactor comprising feeding at least a portion of said reactants to a bottom section of the reactor positioned below a flow distributor plate, passing said portion through the flow distributor plate, passing said portion through a layer of inert particles positioned above and preferably in contact with said flow distributor plate, passing said portion through a catalyst layer comprising a particulate catalyst, said catalyst layer being positioned above and in contact with said layer of inert particles, wherein the reactants react to form a product stream, collecting said product stream via collecting means positioned above said catalyst layer. The invention also relates to a reactor assembly. The catalyst is a core-shell catalyst which is manufactured by graft polymerizing aromatic vinyl compounds onto the hydroxylated and functionalized core particles, followed by sulfonation.

### CROSS REFERENCE TO RELATED APPLICATIONS

This application is a National Stage application of PCT/EP2022/085306, filed Dec. 12, 2022, which claims the benefit of European Application No. 21215714.3 and of European Application No. 21215716.8, both filed Dec. 17, 2021, both of which are incorporated by reference in their entirety herein.

### BACKGROUND

The present invention relates to a method for carrying out a catalysed chemical reaction using one or more liquid reactants in an upflow reactor comprising feeding at least a portion of said reactants to a bottom section of the reactor positioned below a flow distributor plate, passing said portion through the flow distributor plate, passing said portion through a catalyst layer comprising a particulate catalyst wherein the reactants react to form a product stream and collecting said product stream via collecting means positioned above said catalyst layer.

The present invention further relates to a reactor assembly for carrying out such a method.

The present invention in particular relates to a method for the manufacture of bisphenol A based on reacting acetone and phenol.

It is known to carry out chemical reactions in an upflow reactor. For example WO 2018/042375 discloses the use of an upflow reactor for producing a dihydroxy compound as well as to a method for producing a dihydroxy compound. The upflow reactor for producing a dihydroxy compound of WO 2018/042375 comprises a vessel, a catalyst bed disposed in said vessel, a distributor in fluid communication with an inlet through which reactants are introduced to said distributor, said distributor being disposed at a lower end of said vessel and comprising distributor perforation(s) disposed in said distributor, at least part of which distributor perforations are in a direction facing away from said catalyst bed; and a collector through which said product dihydroxy compound is removed, said collector being disposed at an upper end of said vessel.

An upflow reactor for the manufacture of dihydroxy compounds such as bisphenol A is also disclosed in WO 2004/033084.

WO 2020/099285 discloses a method for manufacturing a bisphenol compound comprising reacting a phenol and a ketone in the presence of a catalyst comprising particles having a core and a shell, wherein the shell comprises an ion exchange resin covering the core at least in part and wherein the core has a density that is higher than the density of the ion-exchange resin, wherein the core of the particles has a density of at least 2500 kg/m3. This reference also discloses a method for manufacturing a bisphenol compound, comprising reacting a phenol and a ketone in the presence such a catalyst and wherein the reaction is performed in an up-flow reactor. The present inventors found that the mechanical stability of the core-shell ion exchange resin catalyst in presence of the reaction mixture at reaction temperature might be compromised under pressure or fluidization in the reactor. In particular, the inventors considered that the bond between the shell and the core is relevant to the long-term stability of the catalyst.

US 2002/0128531 discloses an oligomerization process for the production of higher aliphatic olefins. In the process, a liquid oligomerization feed stream comprising lighter aliphatic olefins is passed to a reactor vessel. The liquid oligomerization feed stream is transported upwardly in the reactor vessel against gravity through a fixed bed of solid oligomerization catalyst under oligomerization conditions.

WO 97/34688 discloses a reactor system for conducting chemical reactions in which a reactor is operated in an upflow mode with a fixed bed catalyst and randomly distributed reactor packing therein. The reactor system and the process in which it is used exhibit plug flow behaviour and are amenable to employing lightly cross-linked ion exchange resin catalysts.

The contents of WO 2020/099285, WO2018/042375 and WO2004/033084 are incorporated herein by reference

### SUMMARY

An advantage of operating a reactor in upflow, in particular for the manufacture of bisphenols such as bisphenol A is that it may allow for higher throughputs, usually expressed in terms of the weight hourly space velocity (WHSV). The WHSV for down-flow reactors is generally about 1.0 for the reason that if a higher pressure is applied on top of the catalyst bed, the bed may compress resulting in a higher pressure drop over the catalyst bed. The WHSV is defined as the weight of feed flowing per unit weight of the catalyst per hour. Thus the WHSV may be defined as ton/hour of feed per ton of catalyst, thus having the unit [1/hr].

The present inventors found that if the flow of reactants through an upflow reactor is increased, the catalyst bed may become (partially) fluidized and/or may show uneven flow patterns like channeling of the reactant feed through the catalyst bed. Channeling is a condition of flow wherein portions of the catalyst bed may be short-circuited and not contacted properly by the fluid in a uniform and consistent manner. An uneven flow through the catalyst bed, such as channeling, and/or fluidisation of the catalyst bed may result in fluctuations of the product mixture composition and generally results in a lower conversion of the reactants and accordingly less efficient use of the catalyst. It was in particular found that distribution plates generate high fluid velocities at or near the openings of such plates. These local fluid velocities are generally higher than the velocity needed for fluidisation while the overall fluid velocity, calculated as the volumetric flow divided by the surface area of the reactor is well below such fluidisation velocity. Thus, even though the overall fluid velocity may not result in fluidisation of the catalyst bed, localised fluidisation occurs and manifests itself for example by means of channeling through or back mixing of the catalyst particles. In case of channeling the feed is less in contact with the actual catalyst thereby adversely affecting the conversion of the reactants and/or the selectivity of the intended product (if applicable). For example, in case of a process to manufacture bisphenol A on the basis of acetone and phenol the channeling will result in a lower acetone conversion and/or the product mixture may comprise more bisphenol isomers.

It is therefore an object of the present invention to provide for a reactor assembly and a method that allows the operation in an upflow reactor wherein uneven flow and/or channeling is avoided or at least reduced to a minimum.

It is another object of the invention to provide for an upflow reactor assembly and a method wherein the catalyst bed has improved stability and can operate at improved weight hourly space velocities.

It is yet a further object of the invention to provide for an upflow reactor assembly and a method wherein the catalyst bed has improved stability, can operate at improved weight hourly space velocities and wherein the catalyst itself is stable in the sense that it maintains its catalytic activity for a long time, i.e. has an improved long-term stability.

One or more of the aforementioned objects are met, at least in part in accordance with the invention which relates to a method for carrying out a catalysed chemical reaction using one or more liquid reactants in an upflow reactor comprising:
feeding at least a portion of said reactants to a bottom section of the reactor positioned below a flow distributor plate,
passing said portion through the flow distributor plate,
passing said portion through a layer of inert particles positioned above and preferably in contact with said flow distributor plate,
passing said portion through a catalyst layer comprising a particulate catalyst, said catalyst layer being positioned above and in contact with said layer of inert particles, wherein the reactants react to form a product stream,
collecting said product stream via collecting means positioned above said catalyst layer, wherein,
the upflow reactor is operated at a weight hourly space velocity of at least 1.5, preferably at least 1.7, more preferably from 2.0-5.0, and
the inert particles have a density of at least 2000 kg/m3 and an average particle size of from 500 to 5000 μm, preferably from 500 to 3000 μm, more preferably from 600 to 1500 μm and
the particulate catalyst comprises or consists of a core-shell catalyst obtained or obtainable by the method disclosed herein,
the height of the layer of inert particles is at least 40 times the average particle size of the inert particles.

### BRIEF DESCRIPTION OF THE DRAWINGS

The above and other advantages and features of this disclosure will become more apparent by describing in further detail exemplary embodiments thereof with reference to the accompanying drawings, in which:

FIG. 1 illustrates a possible reaction taking place during silylation with 3-(trimethoxysilyl) propyl methacrylate (MPS);

FIG. 2 illustrates a possible reaction taking place during graft polymerization of polystyrene onto functionalized core particles;

FIG. 3 illustrates a possible reaction taking place during sulfonation of a grafted polymerized styrene;

FIG. 4 are Scanning Electron Microscope (SEM) images of a grafted polymerized styrene samples up to third cycle of stability test of Example 1;

FIG. 5 shows thermal gravimetric analysis (TGA) data for Example 1 after complete removal of unbound polystyrene (PS) by Soxhlet extraction and after each cycle of adhesion stability test;

FIG. 6 shows EDS mapping of a cross-sectional image for polymerized styrene grafted onto a glass bead sample of Example 1;

FIG. 7 shows SEM-EDAX analysis of samples of Example 2 before and after sulfonation;

FIG. 8 shows a comparison of catalytic performance of the manufactured core-shell catalyst of Example 2 with commercially available benchmark catalyst;

FIG. 9 shows selectivity towards p,p-BPA formation with respect to o,p-BPA of Example 2;

FIG. 10 shows a comparison of performance of a comparative example IER catalyst vis-à-vis a core shell catalyst for bisphenol A (BPA) conversion up to sixth cycle; and

FIG. 11 is a partial cross-sectional view of an exemplary reactor assembly.

### DETAILED DESCRIPTION

The present inventors found in particular that the openings in a distributor plate may give rise to a locally increased speed of the reactants potentially causing uneven flow patterns, back-mixing and disruptions or channeling in the layer on top of the distributor plate. Accordingly the present invention requires that a layer of inert particles is positioned on top of the distributor plate and between the distributor plate and the catalyst bed or catalyst layer. The function of the layer of inert particles is to homogenise the flow of the reactants as much as possible so that the reactants will move through the catalyst bed in a plug flow mode or at least substantially in plug flow mode. As a result the catalyst bed or catalyst layer stability will be improved and back mixing and channeling through the catalyst layer is reduced to a minimum.

The distributor plate should allow for the reactants to flow in an upwards manner while at the same time it should act as a support for the layer of inert particles. Thus, the openings or holes in the distributor plate should not be too big as otherwise the inert particles may travel through the distribution plate into the bottom section of the reactor. On the other hand, the holes or openings should also not be too small as that would increase the pressure required to pass the reactants through the distributor plate. To the extent the inert particles contain a fraction of fines that may have a size smaller than the openings of the distributor plate this fraction should be kept as low as possible, preferably at most 15 wt. % based on the weight of the layer of inert particles. Preferably the distributor plate is a slotted plate with a plurality of openings having a size smaller than the average particle size of the inert particles and preferably having a size from 50 to 500 μm, preferably from 150 to 300 μm. The distributor plate preferably has a porosity of 10-50%, preferably from 15-35%, wherein the porosity is defined as the percentage of surface area through which reactants can flow relative to the surface area in flow direction of the distributor plate. Put differently, the porosity refers to the total surface area of openings in the distributor plate relative to the total surface area of such a plate in flow direction.

The inert particles constituting the layer of inert particles are particles having a relatively high density and a preferred (average) particle size. Thus, the present inventors have found it essential that the particles have a density of at least 2000 kg/m3 and an average particle size of from 500 to 5000 μm. The higher the density of the particles, the more resistance they will provide to the flow coming from the distributor plate and the better they are capable of evening out any uneven flow patterns. The present inventors found that particles in the range of from 500 to 5000 μm should be used for the present invention. A preferred average particle size may be from 500 to 3000 μm, more preferably from 600 to 1500 μm. As explained particles with a diameter smaller than the diameter of the holes in distributor plate may migrate to the bottom part of the reactor while particle having a too large particle size become less effective in stabilising the flow of reactants. The particle size distribution may be broad or narrow and may be mono-modal or multimodal such as bimodal. In an embodiment the layer with inert particles is a combination of two or more layers with mutually different average particle size stacked on top of each other, for example with decreasing average particle sizes. The average particle size of the inert particles may be determined using known methods, a sieving method being preferred. It is preferred that the inert particles are obtained by a sieving method so that there is no fraction of particles having a size smaller the openings of the distributor plate. Thus, it is preferred that there is no fraction of inert particles having a size smaller than 500 μm or 600 μm (as the case may be). Put differently it is preferred that at least 95 wt. %, preferably at least 99 wt. %, more preferably at least 99.9 wt. % of the inert particles has a particle size from 500-5000 μm, preferably from 600-3000 μm. The term “particle size” means the diameter in case of spherical particles.

The height of the inert layer in accordance with the invention is at least 40 times the average particle size, typically 40-80, such as 40-60 times the average particle size (diameter) of the inert particles. The present inventors found that this height is sufficient for homogenising the flow patterns and to reduce any local flow deviations to a minimum thereby generating a substantial plug flow mode. In practice the present inventors found that the height of the layer of inert particles is preferably at least 2.0 cm, preferably at least 2.5, more preferably at least 3.0 cm. The upper limit for the height of the bed is less critical although it should not be excessive as that may require a larger reactor and/or the use of more energy to transport the reactant flow through the bed. Typically therefore the height of the inert layer is at most 150 times, preferably at most 100 times the average diameter of the inert particles. In practice a bed height for the inert particles may be from 2.0-15 cm, preferably from 3.0-10 cm. The term plug flow mode in the context of the invention specifically means that the flow-velocity of the reactants is substantially constant over the cross section of the reactor and that there is no or very limited back mixing, wherein the term substantially constant means that the flow-velocity varies at most 5% with respect to the average flow-velocity.

It is preferred that the inert particles comprise, essentially consist or consist of particles selected from sand particles, glass particles, ceramic particles, diatomaceous earth particles, inert metal particles and combinations of at least two hereof. Sand, in particular silica sand, is the preferred material. By means of known sieving techniques the desired particle size and/or particle size distribution for the inert particles can be obtained. The density of the inert particles may be from 2000-5000 kg/m3, preferably from 2100-3500 kg/m3, 2300-3000 kg/m3, 2300-2800 kg/m3. It is preferred that only a single type of material is used, i.e. that the density of each particle, regardless of its particle size, is substantially the same meaning that the density of each particle is at most 10%, preferably at most 5% larger or smaller than the average density for all of the particles.

Alternatively the inert particles may comprise first and second types of materials wherein the first material has an average particle size smaller than an average particle size of the second material and a density that is higher than the density of the second material.

In order to further increase the WHSV while operating the catalyst bed in a fixed bed mode, i.e. an operational mode wherein no channeling and/or fluidisation of the catalyst bed occurs the present have found it advantageous to use a core-shell catalyst manuafctured in accordance with the method disclosed herein below. Such catalysts have a higher density and accordingly provide more resistance to channeling or fluidisation as compared to current commercial ion-exchange resin particles typically being based on a polymeric material such as polystyrene and typically having a density in the range of from 800-1300 kg/m3.

The core-shell catalyst catalyst according to the present invention are manufactured with a method comprising the steps of
a. providing core particles comprising or consisting of glass particles,
b. functionalizing at least part of the surface of said core particles with a functionalizing agent thereby forming functionalized core particles,
c. graft polymerizing at least one of aromatic vinyl compounds onto the functionalized core particles thereby forming core-shell particles wherein the core is comprised of the core particles and the shell is comprised of graft polymerized aromatic vinyl compounds and
d. activating the shell, wherein prior to step b) the core particles are hydroxylated.

### Core Particles

The core particles of the core shell catalyst serve two main purposes. First, the use of core particles allow the increase of the overall density of the catalyst particle, which is especially desired, in the case for use of the core-shell catalyst in an upflow reactor wherein a fixed bed operation is desired. Secondly, the core particles provide a substrate onto which a relatively thin layer of ion exchange resin can be graft polymerized. The use of such a thin layer was found to contribute to an improved selectivity. Thus, compared to commercially available ion-exchange resin beads, such core shell particles allow for a higher selectivity towards the formation of p,p-bisphenol A upon reacting acetone and phenol in the presence of said catalyst.

The core particles comprise or consist of glass particles.

Glass in particular includes silicate glass such as natural or synthetic quartz glass, boro-silicate glass, soda-lime-silicate glass, lead glass and alumino-silicate glass.

In the context of the present invention, the core particles are inert towards the reactants, reaction products and by-products typically used for the manufacture bisphenols, such as acetone, phenol and bisphenol A in particular. The core particles are preferably substantially spherical in shape meaning that a ratio Dₘₐₓ/Dₘᵢₙ is in the range of from 0.85-1.15, preferably from 0.95-1.05, more preferably 0.98-1.02, wherein Dₘₐₓ is a maximum diameter measured on the (core) particle and Dₘᵢₙ a minimum diameter measured on the (core) particle. Generally Dₘₐₓ/Dₘᵢₙ will be from 0.99-1.01. Most preferably, Dₘₐₓ/Dₘᵢₙ is 1.00 meaning that the core particles are perfect spheres having monodispersed distribution.

The core particles have an average particle diameter of 200-2000 μm, preferably 500-1000 μm as determined with microscopy. The average particle diameter of the core particles can be determined by commonly known techniques such as by microscopy methods, laser diffraction and sieving techniques. A preferred microscopy method is SEM (Scanning Electron Microscopy). The skilled person is well aware of such techniques. For the avoidance of doubt, it is noted that the average particle diameter is to be understood as the number based average. The average particle diameter may be determined by measuring, using microscopy such as SEM, the individual particle diameter of 25 particles followed by calculating the numerical average.

In the context of the invention, the core particles have a density of 2000 kg/m³ or more, preferably 2500 kg/m³ or more, such as 3000 kg/m³ or more. Suitably, the core particles have a density of 10000 kg/m³ or less, preferably 9000 kg/m³ or less, such as 8000 kg/m3 or less. Preferably, the density of the core particles is from 2000-4000 kg/m³.

### Hydroxylation

In accordance with the invention, the core particles, preferably glass beads, are first hydroxylated prior to being functionalized. Hydroxylation increases the amount of hydroxyl groups on the surface of the glass which allows for a more effective functionalization which in turn results in an improved adherence of the shell to the core.

Hydroxylation can be effected by treating the surface of the particles with an acidic or a basic solution. A preferred basic solution comprises a strong base such as sodium-hydroxide, potassium-hydroxide or cesium-hydroxide. A preferred acidic solution comprises a strong acid such as for example sulphuric acid, nitric acid, hydrochloric acid or phosphoric acid. A particularly usable solution is known as piranha solution which is a solution comprising sulphuric acid and hydrogen peroxide in molar ratio's of from 3:1 to 7:1. Other aqueous solutions of inorganic or organic bases may be useful provided they result in an increased amount of hydroxyl groups on the surface of the core particles.

Hydroxylation is effected by contacting the surface of the glass with the acidic or basic solution as described above for a certain amount of time and preferably at an increased temperature, i.e. temperatures of from 40-90° C. The time of hydroxylation depends on the temperature as well as on the strength of the base or acid that is used.

Following the step of contacting the glass surface of the core particles with the hydroxylation solution the glass surface of the core particles is preferably dried, for example by first washing the core particles with water and thereafter by acetone or a C1-C4 alcohol. The present invention is not strictly limited to this however and as long as an amount of hydroxylation is maintained any method of drying (if any) may be applied.

In order to check whether hydroxylation has occurred the skilled person has at its disposal known analytical techniques. For example, the FT-IR based ‘Philips Lighting method’ can be applied, the details of which can be found in the publication “Bert Sloots, Measuring the low OH content in quartz glass, Vibrational Spectroscopy, Volume 48, Issue 1, 2008, Pages 158-161, ISSN 0924-2031”.

In addition to analytical techniques the present inventors observed that if no or insufficient hydroxylation took place that there was an insufficient amount of grafting of vinyl aromatic polymer onto the particles which could be visually observed in that the particles would remain substantially transparent. To the contrary, if there was sufficient hydroxylation then there would be sufficient grafting of polymer resulting in the glass core particles no longer being transparent.

For the avoidance of doubt it is noted that the present invention is directed in step a) to the provision of core particles which is then followed by hydroxylation of the same prior to said hydroxylated particles being functionalised. The present invention however is also directed to the provision in step a) of hydroxylated core particles, which implies that hydroxylation has taken place prior to the hydroxylated core particles being functionalised.

### Functionalization of the Core Particles

At least part of the surface of the core particles is functionalized with a functionalizing agent thereby forming functionalized core particles. The functionalizing agent preferably forms a covalent bond with the surface of the core particle substrate thereby forming a functionalized coating on the core particle. Other types of bonding, such as ionic bonding, van der Waals bonding or hydrogen-bridge bonding may be suitable, yet are less preferred.

### Functionalizing Agent

The functionalizing agent is preferably a silane, which typically have the ability to form a chemical bond between organic and inorganic materials. In the present invention, the silane-functionalizing agent is represented by the following general formula (1),

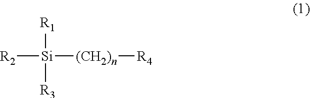

wherein,
R₁, R₂ and R₃ may be the same or different, and are independently selected from the group consisting of hydrogen, halogen, alkyl, haloalkyl, alkylene, alkynyl, aromatic groups, substituted aromatic groups, heteroaromatic groups, ester, ether, acrylate, allyl, alkoxy, alkynoxy, aryloxy, acyloxy, methacrylate, isocyanate, urethane, carbamate, epoxy, carboxylic acid, carboxylic acid anhydride, carboxylate, hydroxy, thiol, amine, aminoalkyl, arylammo and amide provided that at least one of R₁, R₂ and R₃ is an acrylate, allyl, alkoxy, alkynoxy, aryloxy, acyloxy, methacrylate, isocyanate, urethane, carbamate, epoxy, carboxylic acid, carboxylic acid anhydride, carboxylate, hydroxy, thiol, amine, aminoalkyl, arylammo and amide,
R₄ is a functional group for reacting with the aromatic vinyl compounds, and
n is between 0-20.

The functionalizing agent comprises at least one functional group for reacting with the core particle and at least one functional group for reacting with one or more of an aromatic vinyl compounds. The functional group for reacting with the core particle typically is a hydrolysable group that can undergo hydrolysis. Following hydrolysis, a reactive silanol group is formed, which can condense with other silanol groups, for example, those on the surface of siliceous fillers, to form siloxane linkages. The other functional group for reacting with one or more of an aromatic vinyl compounds is a non-hydrolysable organic radical that may possess a functionality that imparts desired characteristics.

The functional group for reacting with the core particles is selected from the group consisting of allyl, alkoxy, alkynoxy, aryloxy, acyloxy, acrylate, methacrylate, isocyanate, urethane, carbamate, epoxy, carboxylic acid, carboxylic acid anhydride, carboxylate, hydroxy, thiol, amine, aminoalkyl, arylamine and amide, and combinations of two or more of the foregoing. For the functionalizing agent according to structure (1), at least one of R₁, R₂ and R₃ is selected from the above functional groups for reacting with the core particles. In a preferred embodiment of the invention, at least one of R₁, R₂ and R₃ is selected from alkoxy, alkynoxy, aryloxy, acyloxy and combinations of two or more of the foregoing. In a particular preferred embodiment of the invention, R₁, R₂ and R₃ are selected from alkoxy, alkynoxy, aryloxy, acyloxy and combinations of two or more of the foregoing.

The functional group for reacting with one or more of the aromatic vinyl compounds is selected from the group consisting of organic groups comprising ethylenically unsaturated groups, acrylate groups, methacrylate groups, aldehyde groups, amine groups, azide groups, alkyl groups, alkenyl groups, alkinyl groups, aryl groups, aralkyl groups, cycloalkyl groups, cycloalkylene groups, hydroxyl groups, carboxyl groups, dipodal silane groups and combinations of two or more of the foregoing groups. For the functionalizing agent according to structure (1), R₄ is selected from at least one of the above functional groups for reacting with one or more of the aromatic vinyl compound. In a preferred embodiment of the invention, the R₄ is selected from organic groups comprising ethylenically unsaturated groups, acrylate groups, methacrylate groups, aldehyde groups and combinations of two or more of the foregoing.

For the functionalizing agent according to structure (1), n denotes the linker length between the organic functionality and the silicon atom. The linker length is an important factor for the physical property and reactivity of the silane-functionalizing agent. Preferably, the linker length, n=0-5, more preferably n=0-3 and most preferably n=3, a consequence of the fact that the propyl group is synthetically accessible, and has good thermal stability and facilitates chemical reactivity.

### Graft Polymerization

In accordance with the invention, after formation of the functionalized core particles, one or more aromatic vinyl compounds is graft polymerized onto the functionalized core particles, thereby forming core-shell particles wherein the core is comprised of said core particles and the shell is comprised of graft polymerized aromatic vinyl compounds.

The aromatic vinyl compound, in the present invention, comprises styrene and optionally a substituted styrene, preferably selected from the group consisting of alpha-methyl styrene, sulphonated styrene, vinyl toluene, ethyl vinyl benzene, vinyl naphthalene, or a combination thereof. Most preferably, the aromatic vinyl compound is a styrene.

The aromatic vinyl compound(s) react with the functional group R₄ of the silane-functionalizing agent according to structure (1), through a graft polymerization reaction. This result in the formation of covalent bonds between the in-situ polymerized aromatic vinyl compounds and the functionalizing agent. Through the process, the inventors have been able to manufacture graft polymerized aromatic vinyl compound(s), which, via the functionalizing agent is covalently bonded to the core particles. The layer comprised of the polymerized aromatic vinyl compound, together with the coating consisting of the functionalizing agent, forms the shell of the core shell particle.

In a preferred embodiment, the core particles comprises or consists of glass particles and the shell comprises or consists of graft-polymerized styrene. Styrene is graft polymerized onto functionalized glass particles, preferably through in-situ emulsion polymerization method. Emulsion polymerization method is well known to a person skilled in art. A suitable reactor to perform emulsion polymerization could be a continuous/batch stirred tank reactor or even a fluidized bed reactor. Alternatively, a bulk polymerization can be performed using hot nitrogen (or any other inert gas) to heat and fluidize the functionalized core particles. Functionalized core particles are initially charged into the reactor and fluidized with hot nitrogen preferably at 70 to 90° C. temperature. Then the styrene and the other monomer(s) can be introduced into the reactor at regular intervals to wet the functionalized core particles. As the particles are fluidized and heated with hot nitrogen, the adhered monomer mixture would polymerize to form a shell layer over the functionalized core particles. This process is repeated until the desired shell thickness is achieved over core particles.

Functionalized core particles are initially charged into the reactor and fluidized with hot nitrogen preferably at 70 to 90° C. temperature. Later, the styrene and the other monomer(s) can be introduced into the reactor at regular intervals to wet the functionalized core particles. As the particles are fluidized and heated with hot nitrogen, the adhered monomer mixture would polymerize to form a shell layer over the functionalized core particles. This process is repeated until the desired shell thickness is achieved over core particles.

A procedure to make core-shell particles is described by Gu et al. (Journal of Colloid and Interface Science 2004, 272(2), 314-320). Although the original article applies this dispersion polymerization based method to prepare nano-sized core-shell particles (800-1500 nm), the present inventors have found that the method may also be applied to prepare micron sized core-shell catalyst particles after surface activation of the micron sized particles (200-2000 μm).

Additionally, the graft polymerization is preferably carried out in the presence of one or more of a cross-linking monomer. The crosslinking monomers include aromatic crosslinking monomers such as divinylbenzene, divinyltoluene, trivinylbenzene, divinyl chlorobenzene, diallyl phthalate, divinylnaphthalene, divinyl xylene, divinylethylbenzene, trivinyl naphthalene and polyvinylanthracenes; and aliphatic crosslinking monomers such as di- and polyacrylates and methacrylates exemplified by trimethylolpropane trimethacrylate, ethylene glycol dimethacrylate, ethylene glycol diacrylate, neopentyl glycol dimethacrylate and pentaerythritol tetra- and trimethacrylates, and trivinylcyclohexane. The crosslinking monomer is preferably present at levels from about 0.1 to about 20 weight percent of the total monomer, and more preferably from about 1 to about 10 weight percent of the total monomer. Preferred crosslinking monomers are; aromatic crosslinking monomers, and particularly preferred is divinylbenzene. In the obtained polymer shell, the degree of cross-linking is preferably from 1-4%, more preferably less than 2% degree of crosslinking.

### Activation

After graft polymerization of the aromatic vinyl compound(s) onto the functionalized core particles, the resulting core-shell particles are activated. Activation of the shell provides the core-shell particles with catalytic activity. The activation comprises sulfonating at least part of the graft polymerized aromatic vinyl compound using a sulfonating agent. The introduction of sulfonic acid groups not only yields catalytically active functional groups but also accomplishes sulfone crosslinking of the polymer in which the aromatic ring contains more than one sulfonic acid group per ring. In a preferred embodiment, the catalyst comprises sulfonic acid functional groups.

Optionally, the shell can additionally comprise an attached promoter, preferably a mercaptan compound, which is attached to catalyst by reacting with sulfonic acid functional groups. A mercaptan compound refers to a compound having a free form of SH group in the molecule. As the mercaptan, an alkyl mercaptan can be used (which may be a non-substituted alkyl mercaptan or an alkyl mercaptan having at least one substituting group, such as a carboxylic group, an amino group, a hydroxyl group, etc.). Examples of non-substituted alkyl mercaptan include methyl mercaptan, ethyl mercaptan, n-butyl mercaptan, and n-octyl mercaptan. Examples of substituted alkyl mercaptan include mercaptocarboxylic acids (such as thioglycolic acid and 3-mercaptopropionic acid), aminoalkane thiols (such as 2-amino ethane thiol and 2,2-dimethyl thiazolidine), and mercaptoalcohols (such as mercaptoethanol). Among these, the non-substituted alkyl mercaptans are preferred in terms of the promoting action. These mercaptans may be used singly or in combination. The promoter is attached to the resin forming the shell of the core-shell catalyst. Presence of an attached promotor allows the manufacture of bisphenol without the need for addition of a separate promoter. Such technology is preferred as it reduced the presence of sulfur containing species in the final product such as in particular bisphenol A.

In accordance with the invention, the shell of the core-shell particles need not necessarily cover the core completely. For example, the shell may cover 50% or more of the surface area of the core, such as 60% or more, 70% or more, 80% or more, 90% or more, or 95% or more. Preferably, the core of the core-shell particles is completely covered with shell material.

Preferably, the shell of the core-shell catalyst has an average catalyst layer thickness 10-500 μm, preferably from 100-300 μm. The average layer thickness of the shell of the core-shell particles can be determined by, e.g., commonly known microscopy methods, automated imaging, laser diffraction and sieving techniques. A preferred method is SEM (Scanning Electron Microscopy). For the avoidance of doubt, it is noted that several ways of determining the shell thickness exist and that a skilled person will have no difficulty in accurately determining the average shell thickness with the average being a numerical average. A convenient method involves first measuring the average diameter of the core particles prior to functionalization. Then after graft polymerisation the average particle diameter is measured again and the average shell thickness can be calculated on the basis of the difference between the average diameter of the core particles and the average diameter of the core-shell particles.

### Manufacture of Bisphenol Using Core-Shell Catalyst

In a further aspect, the invention is directed to a method for the manufacture of a bisphenol comprising reacting a phenol and a ketone in the presence of the core-shell catalyst as prepared by the above-mentioned steps. In the said method, the reaction is performed in an up-flow reactor, wherein, the reactants are fed to the reactor at an inlet and bisphenol is extracted at an outlet at a position higher than the inlet, or in a down-flow reactor, wherein the reactants are fed to the reactor at an inlet and bisphenol is extracted at an outlet at a position lower than the inlet.

The bisphenol may, for instance, be bisphenol A (2,2′-bis(4-hydroxyphenyl)propane), bisphenol S (4,4′-sulphonyldiphenol), or bisphenol F (4,4′-dihydroxydiphenylmethane). Preferably, the bisphenol is bisphenol A.

Typically, the reaction temperature at which the reaction between the phenol and ketone is carried out is from 40-150° C., preferably 60-110° C., more preferably 50-80° C. The reaction is performed continuously. The upflow reactor may be a single upflow reactor, or maybe two or more such reactors connected in series. Alternatively or in addition the the upflow reactor comprises multiple reaction zones, each zone separated from another zone by means of a collection zone above a catalyst bed. For example the upflow reactor may comprise from 2-10 zones wherein each zone has a distributor plate in contact with a layer of inert particles, which in turn is in contact with a layer of catalyst in the manner as disclosed herein.

In yet another aspect, the invention is directed to the use of the catalyst in a catalyst bed of an up-flow or a down-flow reactor for the manufacture of a bisphenol. Preferably the catalyst bed is a fixed bed. The catalyst of the invention increases bed stability, minimizes back mixing, and allows to operate the reactor at higher space velocity. Furthermore, the invention is directed towards the use of the catalyst for the manufacture of bisphenol A by reacting phenol with acetone for increasing the selectivity towards formation of p,p-bisphenol A.

For the purpose of the invention the catalyst particles preferably have an average particle size, in use, of from 500 to 1500 μm. The particle size, in use, may be from 600-1200 μm such as from 900-1100 μm. The term “in use” refers to the particle size of the catalyst when it is used in carrying out the intended chemical reaction. In particular ion exchange resins may shrink or expand depending on the reaction medium that they are exposed to and/or depending on the medium in which they are supplied to the end-user. To determine the “in use” diameter a sample of catalyst can be taken from the reactor and analysed using known means for determining the particle size such as optical microscopy or scanning electron microscopy. For the avoidance of doubt it is noted that the present invention is not limited to catalysts that show such shrinkage or expansion. The catalyst may have a particle size distribution and accordingly it is preferred that at least 95 wt. %, more preferably at least 99 wt. %, more preferably at least 99.9 wt. % of the catalyst particles has a particle size from 500 to 1500 μm, preferably from 600-1200 μm. Typical core-shell ion-exchange resin catalysts are comprised of substantially spherical beads so that accordingly it is preferred that the average diameter of the catalyst is from 500 to 1500 μm, preferably from 600-1200 μm. Likewise it is preferred that at least 95 wt. %, more preferably at least 99 wt. % or 99.9 wt. % of the catalyst particles has a diameter from 500 to 1500 μm, preferably from 600-1200 μm.

The present invention allows a chemical reaction to be carried out at a higher weight hour space velocity (WHSV). The WHSV is defined as the weight of feed flowing per unit weight of the catalyst per hour. The present invention allows chemical reactions to be carried out at higher WHSV as compared to down-flow reactors while maintaining a desired conversion of the reactants. That is the present invention allows for a chemical reaction to take place at a WHSV of at least 1.0, such as at least 1.1, at least 1.2 or at least 1.5. Preferably the WHSV is from 1.1-10, 1.3-8 more preferably from 1.5-5.

The present invention is preferably directed at a method for the manufacture of a bisphenol, in particular bisphenol A, by reacting ketone with phenol. More specifically the present invention is directed at a method for the manufacture of bisphenol A by reacting acetone and phenol in the presence of an ion-exchange resin catalyst. In such method the product stream comprises bisphenol A, phenol, acetone, water and by products, wherein the amount of bisphenol A is from 15-35, preferably 20-30 wt. % based on the weight of the product mixture. Likewise the present invention is preferably directed at a reactor assembly for the manufacture of bisphenol A by reacting acetone and phenol in the presence of an (acidic) ion-exchange resin catalyst.

The present invention further relates to a reactor assembly for carrying out a catalysed chemical reaction using one or more liquid reactants in upflow comprising:
feeding means for feeding at least a portion of said reactants to a bottom section of the reactor, said feeding means being positioned below a flow distributor plate,
layer of inert particles positioned above and preferably in contact with said flow distributor plate,
a catalyst layer comprising a particulate catalyst positioned above and in contact with said layer of inert particles,
collecting means positioned above said catalyst layer for collecting a product stream from the reactor,
wherein,
the inert particles have a density of at least 2000 kg/m3 and an average particle size of from 500 to 5000 μm, and
the particulate catalyst comprises or consists of a core-shell catalyst obtained or obtainable by the method disclosed herein,
the height of the layer of inert particles is at least 40 times the average particle size of the inert particles.

The reactor used for the method and the apparatus disclosed herein is typically substantially cylindrical in shape. Substantially cylindrical means that the ratio between the largest diameter and the smallest diameter is from 0.9-1.1.

For the avoidance of doubt it is to be understood that the present invention relates to a method and reactor assembly for carrying out one or more chemical reactions in a liquid phase. Thus, the present method excludes gas-phase reactions. Although the general principle of the reactor assembly will work for gas phase reactions as well the requirements for WHSV, catalyst density, catalyst particle size, inert particle density and inert particle size will be different as will be understood by a skilled person.

All preferred aspects disclosed herein related to the method and/or the catalyst also apply to the reactor assembly.

The present invention will now further elucidated herein on the basis of the following non-limiting examples and Figures.

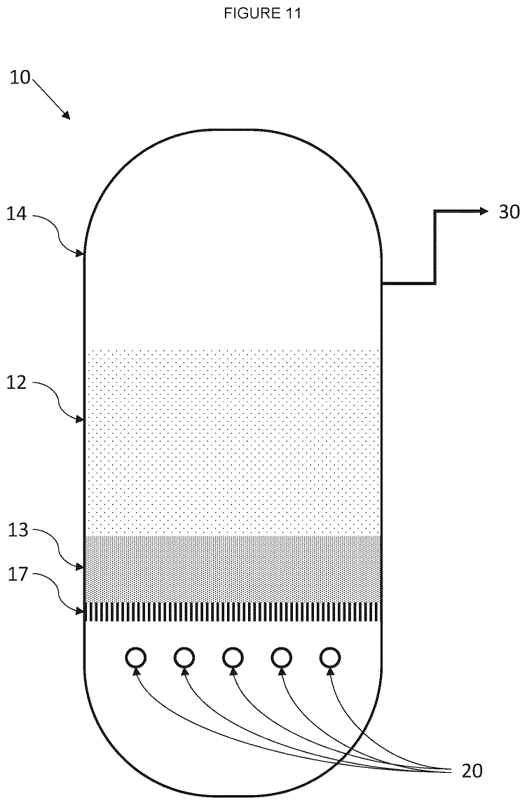

FIG. 11, not to scale, is a partial cross-sectional view of an exemplary reactor assembly 10 of the present disclosure. In general, reactor assembly 10 is a packed bed reactor assembly that is configured and dimensioned to produce dihydroxy compounds (e.g., bisphenols, preferably bisphenol A). Reactor assembly 10 includes a distributor unit 20 having a plurality of distributor pipes (e.g., four distributor pipes) with holes or perforations pointing downward, with the plurality of distributor pipes 20 forming a distributor unit positioned evenly across the cross-section of the vessel 14 of reactor assembly 10.

Above the distributor unit, a distributor plate 17 (e.g., a slotted plate or wire mesh plate with openings having a size from 50 to 500 micrometers, preferably from 150 to 300 micrometers) is positioned to support the layer of inert particles 13 and the catalyst layer 12. The feed to the reactor assembly 10 via distributor pipes 20 of the distributor unit includes phenol and acetone. From the pipes 20 the liquid feed is injected predominantly in a direction away from the distributor plate 17.

The inert layer, i.e. layer with inert particles 13 is positioned on or above distributor plate 17 to help distribute the feed from the distributor unit uniformly to the catalyst layer 12.

The inert layer 13 can comprise, without limitation, sand particles, glass particles, silica particles, metal particles (e.g., metal particles that are inert in the feed mixture, such as, for example, nickel particles and/or titanium particles), or a combination comprising at least one of the foregoing. The height of the inert layer 13 (e.g., sand layer 13) on top of distributor plate 17 in vessel 14 can be on average 2.5 cm.

A lower limit for the height of the inert layer 13 on top of distributor plate 17 can be about 2 cm. However, it is noted that the height of the layer 13 on top of distributor plate 17 can be 4 cm to 5 cm. It is noted that there may be no significant benefit having a height of the layer 13 on top of distributor plate 17 greater than 10 cm.

The particles (e.g., sand particles) of layer 13 can have an average particle size from 700 to 1200 μm.

In use, a catalyst layer 12 can be disposed on the inert layer 13 (layer of inert particles), said catalyst layer 12 including ion exchange resin particles (e.g., acidic ion exchange resin catalyst particles), the ion exchange resin particles optionally comprising an attached promoter (e.g., co-catalyst promoter).

For the avoidance of doubt it is noted that the flow direction in reactor assembly 10 is from bottom to top, i.e. from distributor pipes 20 to collecting means 30.

Collecting means 30 for collecting the product mixture from reactor assembly 10 are positioned above catalyst layer 12. The exact location of said collecting means may vary.

In an embodiment a further product distribution plate (not shown) may be positioned above catalyst layer 12. Such a product distribution plate is optionally in direct contact with the catalyst layer. The porosity of such a distribution plate is preferably the same or higher than the porosity of the distributor plate. Preferably the product distributor plate is a slotted plate with a plurality of openings having a size smaller than the average particle size of the catalyst particles and preferably having a size from 300-1000 μm, preferably from 500-800 μm. The product distributor plate, in particular when in contact with the catalyst layer 12 further supports the catalyst bed stability and may be used to prevent catalyst particles to end up in the collecting means. A combination of several product distributor plates may be used. While the present invention is disclosed herein with reference to an upflow reactor with a single catalyst bed, i.e. a single reaction zone, the present invention also applies to an upflow reactor comprising a plurality of reaction zones, such as for example from 2-10, preferably 3-6 reaction zones. Prefereably in each of the reaction zones the configuration is similar to that disclosed in FIG. 11. Thus in each reaction zone there is a distributor plate 17 on top of which and in direct contact therewith is a layer of inert particles 13 on top of which and in direct contact therewith is a layer with catalyst particles 12

The product mixture typically contains bisphenol A, acetone, phenol, water and impurities or byproducts. The product mixture is further processed in downstream process steps to isolate and purify the bisphenol A product. Such steps are known to a skilled person.

### EXAMPLES 1-7

Experiments were conducted using the configuration of the reactor assembly 10 as shown in FIG. 11. Reactor assembly 10 included a distributor unit having four distributor pipes 20 with holes or perforations pointing downward, with the four distributor pipes of distributor unit 20 positioned evenly across the cross-section of vessel 14.

Vessel 14 had an inner diameter of 800 millimeters (mm). The tangent to tangent height of vessel 14 of 2000 mm was sufficient to contain the catalyst volume of catalyst layer 12 and provided sufficient empty space for proper liquid collection using suitable collecting means 30. Above the distributor unit, a distributor plate 17 was positioned. Distributor plate 17 was a slotted plate with openings having a diameter of 200 μm.

An amount of 0.50 m³ of ion-exchange resin catalyst formed catalyst bed 12.

The ion-exchange resin catalysts for catalyst bed 12 used in the experiments were commercially available 2% cross-linked sulfonated polystyrene which differed in particle size distribution.

Catalyst Type 1 had a poly-disperse particle size distribution with a ratio of D90/D10 of about 1.4 and with an average particle size of about 1065 μm.

Catalyst Type 2 had a mono-disperse particle size distribution with a ratio of D90/D10 of about 1.1 and with an average particle size of about 875 μm.

The particle size and particle size distribution was determined via an image analysis technique.

Table 1 shows results of experiments carried out at different flow speeds, representative for the amount of material flowing through the reactor and accordingly for the WHSV. The feed consisted of a mixture of 5 wt. % acetone and 95 wt. % phenol and catalyst type 2 was used. The sand layer in examples CE1-CE3 consisted of sand particles obtained by sieving of river sand and having a particle size distribution from 0.7 to 1.2 mm, and a bulk density of 1.58 kilogram/liter (kg/l). The height of the sand layer 13 on top of distributor plate 17 in vessel 14 was on average 2.5 cm. The average particle size of the sand particles was about 1000 μm. The density of the sand particles of sand layer 13 was about 2500 kg/m³.

TABLE 1

| | CE1 | CE2 | CE3 | CE4 | CE5 | CE6 |
|---|---|---|---|---|---|---|
| Flow_speed | 0.133 | 0.200 | 0.267 | 0.133 | 0.200 | 0.267 |
| [mm/s] | | | | | | |
| WHSV | 1.0 | 1.5 | 2.0 | 1.0 | 1.5 | 2.0 |
| Acetone | 94.5 | 89.6 | 74.5 | 96.3 | 85.9 | 71.8 |
| conversion | | | | | | |
| [%] | | | | | | |
| ppBPA | 94.0 | 93.7 | 93.7 | 94.0 | 94.6 | 94.0 |
| Selectivity | | | | | | |
| [%] | | | | | | |
| Feed | 55 | 55 | 60 | 55 | 55 | 60 |
| temperature | | | | | | |
| [° C.] | | | | | | |
| Sand layer | yes | yes | yes | no | no | no |
| Catalyst type | 2 | 2 | 2 | 2 | 2 | 2 |

The flow speed corresponds to the upward linear velocity of the flow in the void reactor area (e.g., the area above catalyst bed 12) of vessel 14 and is defined as the volumetric flowrate divided by the cross-sectional area of the vessel 14 of the reactor assembly 10.

The acetone conversion represents the weight percent of acetone that is converted during the reaction and based on measurement of the acetone concentration in the outlet of the reactor.

The p,p-bisphenol A (ppBPA) selectivity represents the weight percentage of p,p-bisphenol A that was produced relative to the total amount of bisphenols.

From Table 1 it can be observed that upon increasing flow speed the acetone conversion decreases when no sand bed (layer of sand) is present. The present inventors believe this was due to an uncontrolled and less homogenous flow through the catalyst layer and may either one or more of catalyst fluidisation and/or back mixing and/or channeling. When a sandbed was used in conjunction with commercially availiable low density catalysts, as per comparative CE1-CE3 it will be aprreciated that a WHSV of 1.5 or even 2 could be obtained. Since the catalyst particles in accordance with the present invention have a much higher density the present inventors consider that even higher WHSV may be achieved.

Table 2 shows the results of experiments that were carried under the same conditions except that catalyst type 1 was used and the concentration of acetone in the feed was 3 wt. % (and the phenol concentration accordingly was 97 wt. %)

TABLE 2

| | CE7 | CE8 | CE9 | CE10 |
|---|---|---|---|---|
| Flow_speed | 0.133 | 0.267 | 0.133 | 0.267 |
| [mm/s] | | | | |
| WHSV | 1.0 | 2.0 | 1.0 | 2.0 |
| Acetone conversion | 98.2 | 93.1 | 96.2 | 88.7 |
| [%] | | | | |
| ppBPA Selectivity | 95.1 | 95.9 | 945.4 | 94.7 |
| [%] | | | | |
| Feed temperature | 55 | 55 | 55 | 55 |
| [° C.] | | | | |
| Sand layer | yes | yes | no | no |
| Catalyst type | 1 | 1 | 1 | 1 |

Table 3 shows the results of further experiments wherein the feed contained fresh acetone and phenol but also a recycle stream containing unreacted acetone and phenol, p,p-bisphenol-A (also known as 2,2-bis(4-hydroxyphenyl)propane or “ppBPA”)), o,p-bisphenol A (also known as 2,4′-isopropylidenediphenol (“opBPA”)) and/or other isomers. Catalyst type 1 was used; the sand layer (if present) was the same as for the Examples in Tables 1 and 2.

The composition of the feed in the experiments CE11/CE13 and CE12/CE14consisted of
3 wt. % of acetone
74.5 wt. % of phenol
12 wt. % of p,p-bisphenol A
3.5 wt. % of o,p-bisphenol A
7 wt. % other isomers including one or more of, 3-(4-hydroxyphenyl)-1,1,3-trimethyl-2H-inden-5-ol (“cyclic dimer 1”); 2,4-bis[1-(4-hydroxyphenyl)isopropyl]phenol (“BPX 1”); 4-(2,2,4-trimethylchroman-4-yl)phenol (“chroman 1”); 4-(2,4,4-trimethyl-3,4-dihydro-2H-chromen-2-yl)phenol (“chroman 1.5”); 1,1′-spirobi[1H-indene]-6,6′-diol,2,2′,3,3′-tetrahydro-3,3,3′,3′-tetramethyl (“spirobiindane”).

The viscosity of this feed was significantly higher compared to the feed in the experiments in Tables 1 and 2.

TABLE 3

| | CE11 | CE12 | CE13 | CE14 |
|---|---|---|---|---|
| Flow_speed | 0.133 | 0.267 | 0.133 | 0.267 |
| [mm/s] | | | | |
| WHSV | 1.0 | 2.0 | 1.0 | 2.0 |
| Acetone conversion | 95.2 | 65.2 | 77.1 | 44.6 |
| [%] | | | | |
| ppBPA Selectivity | 98.4 | 98.8 | 101.2 | 103.2 |
| [%] | | | | |
| Feed temperature | 62.8 | 63.2 | 56 | 63.4 |
| [° C.] | | | | |
| Sand layer | yes | yes | no | no |
| Catalyst type | 1 | 1 | 1 | 1 |

Because of the presence of isomers in the feed, which may be converted into p,p-bisphenol A, the ppBPA selectivity could be over 100%.

Since the feed in the Examples of Table 3 has a significantly higher viscosity (about 2.5 times higher) compared to the Examples in Tables 1 and 2, the settling rate of the catalyst particles in the feed medium was higher, explaining a larger effect on the acetone conversion. Additionally and as mentioned, because this feed also contains products that can be converted by the catalyst into ppBPA (e.g., isomerisation of o,p-bisphenol A) the net calculated ppBPA selectivity % can be above 100%.

By means of computational fluid dynamics simulations the present inventor confirmed that advantageous effect of the sand layer. For a reactor equipped with a tri-slot distributor plate having 1.8 mm width bars with 0.2 mm clearance between them they found that at a WHSV of 2 they observed that without a sand layer significant channeling and/or back mixing occurred in the catalyst layer. When the catalyst layer was put on top of a sand layer of about 5 cm the channeling and back mixing was reduced to a minimum and a more even fluid velocity pattern was observed.

### Core Shell Catalyst Experiments

### Materials Used and Their Sources:

Glass beads were supplied by Retsch (product code 22.222.0004), 3-(trimethoxysilyl) propyl methacrylate (MPS), ethanol, sodium hydroxide (NaOH), styrene, sodium styrene sulfonate (NaSS), potassium persulfate (KPS) and divinyl benzene (DVB) were purchased from Sigma-Aldrich (India) and were used as received.

### Preparation of the Core-Shell Catalyst:

### Step 1: Hydroxylation of Glass Surface—Core Particles

Glass beads (20 g) were added to a two-necked 100 mL round bottom (RB) flask equipped with an overhead stirrer and condenser and 25 mL of 5M NaOH was added to it. The mixture was stirred using an overhead stirrer for the next 24 hours at 90° C. The reaction mixture was cooled to room temperature and subsequently, quenched by adding excess deionized (DI) water. The glass beads were removed and washed with DI water until the pH of the aqueous phase became neutral. Finally, the glass beads were washed with acetone and dried under vacuum at room temperature.

Step 2: Silylation with MPS—Functionalized Core Particles

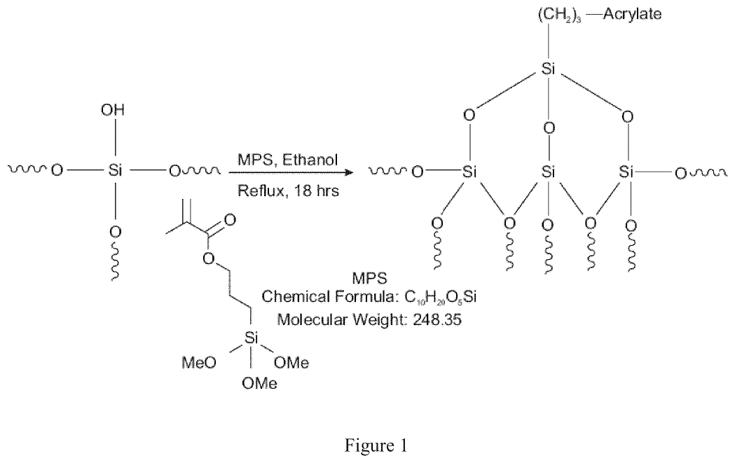

Cleaned and dried glass bead samples (6.0 g) as obtained at the end of step 1 were added to a two-necked 100 mL round bottom flask, equipped with an overhead stirrer and a condenser. MPS (7.1 mL) followed by 20 ml of ethanol was added to the flask. The mixture was stirred under reflux for 18 hours and then the resulting silylate-functionalized glass beads were washed several times (3×50 mL, for 30 minutes with stirring) with ethanol to completely remove any excess or unreacted MPS adhered to the glass bead surface. Finally, the silylate-functionalized glass beads were dried under vacuum at room temperature. The present inventors believe the reaction taking place during the functionalization of this Step 2 is as shown in FIG. 1.

Step 3: Graft Polymerizing Polystyrene onto the Functionalized Core Particles—Shell

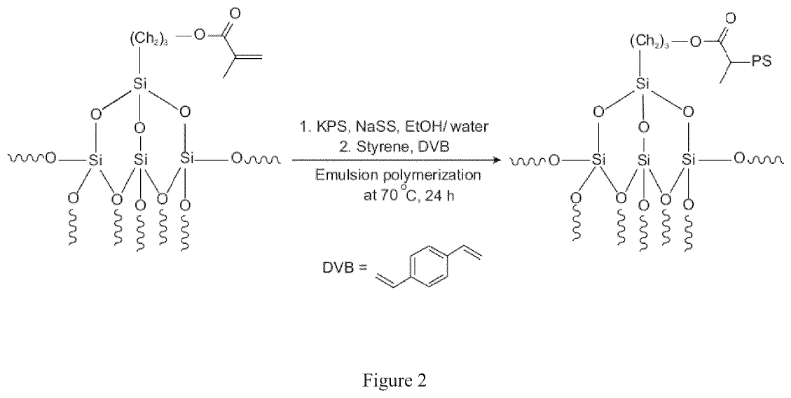

In a 100 mL three necked round bottom flask, KPS (0.27 g) and NaSS (27 mg) was dissolved in water (5.5 mL) and was charged with nitrogen using a purge tube for 15 minutes. Silylate-functionalized glass beads as obtained at the end of Step 2 (5 g) was charged into the RB flask under nitrogen atmosphere. Ethanol (10.2 mL) was added under nitrogen atmosphere and left for purging for another 15 minutes. The mixture was heated to 70° C. and then styrene (5.8 mL) was added to the reaction mixture under nitrogen atmosphere. After 30 minutes of stirring using overhead mechanical stirrer, DVB solution (115 μL) in 2 mL ethanol was added to the reaction mixture. The reaction mixture was then left at 70° C. under nitrogen atmosphere for 24 hours under continuous stirring. After that, the emulsion was removed by repetitive washing with water. The samples were stirred in chloroform (CHCl₃, 70 mL) for 18 hours to extract any free polystyrene (PS) that is not covalently bound to the glass bead surface. Finally, the obtained core-shell particles were washed with methanol to remove any moisture and then dried under vacuum at room temperature. At this stage, the glass bead surface was found to be white in color showing grafting of polystyrene on the glass bead surface. The present inventors believe the reaction taking place during the functionalization of this Step 3 is as shown in FIG. 2.

### Step 4: Sulfonation of the Grafted Polymerized Styrene—Activation

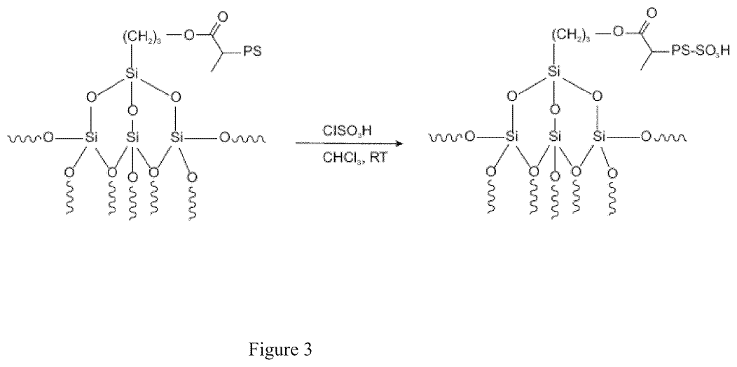

The core-shell particles obtained at the end of step 3 (1.0 g) were sulfonated by immersing the samples in in chloroform (15 mL) at room temperature for one hour under stirring using overhead mechanical stirrer followed by addition of an amount of chlorosulfonic acid (1.3 mole equivalent to the grafted PS content). The reaction mixture was then left for shaking for one hour in a mechanical shaker. After shaking for one hour, the resulting sulfonated core-shell particles were gradiently washed with chloroform (30 mL×3) to remove the excess acid present in the reaction mixture. After washing with chloroform, the sulfonated core-shell particles were further washed with mixture of chloroform and water (20 mL water×3). Finally, the sulfonated core-shell particles was washed with acetone and dried under vacuum at room temperature. The present inventors believe the reaction taking place during the functionalization of this Step 4 is as shown in FIG. 3.

### Comparative Example 1 (without Hydroxylation)

In this example glass beads were silylated with MPS under the same condition as mentioned in Step 2 and Step 3 without however performing the hydroxylation of the glass surface as mentioned in Step 1. The particles obtained at the end of step 3 were examined visually to detect if polystyrene was grafted on the core particles. It was found that the glass beads remained transparent and that no, or at least negligible visible grafting of polystyrene on the surface glass bead surface had occured.

### Catalyst Example 1

In this example, the particles obtained at the end of Step 3 (hydroxylated and polymerized styrene grafted core-shell particles) were analyzed to determine the adhesion stability of the grafted polymerized styrene onto the glass beads. These particles were subjected to Soxhlet extraction using chloroform as solvent to remove any trace amount of unreacted styrene and other ingredients. Unbound PS particles were further removed from the dried mixture by using sieves. The obtained particles were used as test sample particles for adhesion stability test as follows.

Initial weight of the test particles were measured at the onset of the adhesion stability test (as-prepared weight). Then the adhesion stability of these test particles were examined for multiple cycles. One cycle of adhesion stability test represents stirring the test sample particles in the mixture of phenol and acetone (96:4, w/w) at 70° C. for 24 hours, washing with acetone for complete removal of phenol, drying under vacuum, and re-weighing the same. Dried samples after each cycle of adhesion stability test was also characterized by microscopy (SEM image analysis) and thermal gravimetric analysis (TGA) to monitor the adhesion stability of grafted polymerized styrene.

For the SEM analysis, the polymerized styrene grafted glass bead samples were gold coated to around 5 nm in thickness and then was observed in a ZEISS make ‘EVO18’ Scanning Electron Microscope (SEM). A comparison of the surface morphology of original glass beads and polymerized styrene grafted glass beads (collected after multiple cycles of adhesion stability test) allowed the inventors to analyze the uniformity of PS coating around the glass surface and adhesion stability of the same. SEM images of the grafted polymerized styrene samples up to third cycle of stability test is shown in

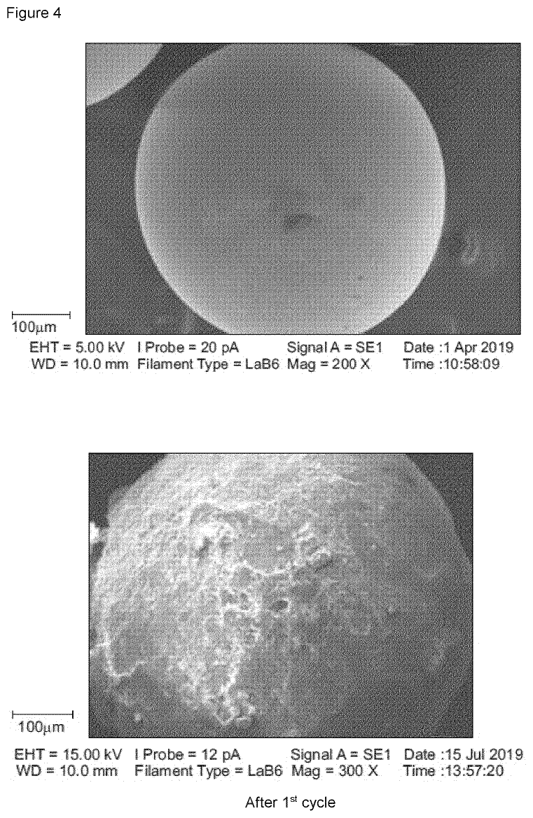

FIG. 4. It was apparent from the images that the in-situ polymerized styrene remain grafted onto the glass bead surface after treated with mixture of phenol and acetone (96:4. w/w) at 70° C. for 24 hours. This demonstrates a high adhesion stability of the grafted polymerized styrene layer even after several cycles.

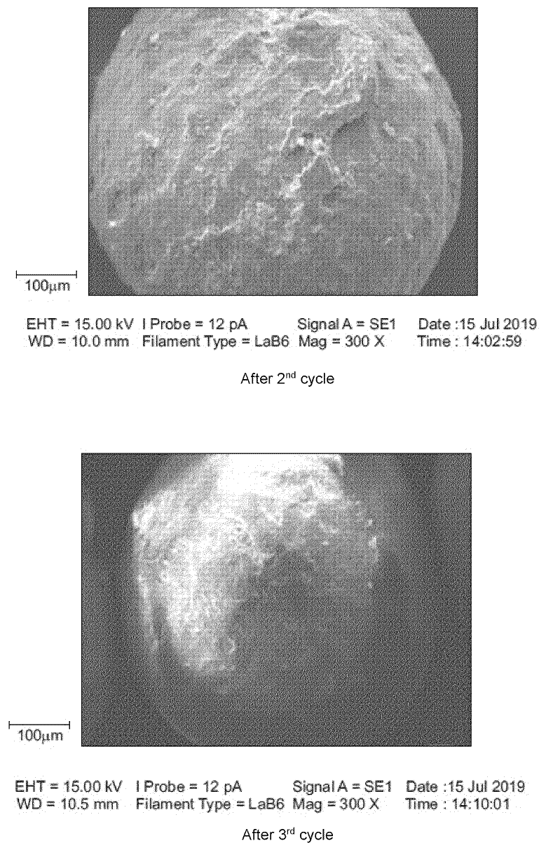

Further, the quantity of polymerized styrene grafted onto the glass bead surface was estimated by thermogravimetric analysis (TGA) by quantifying the weight loss due to grafted PS. The TGA of dried grafted polymerized styrene glass bead sample particles collected after each cycle of adhesion stability test were performed on a TA Instruments TGA Q5000 in air at a heating rate of 20° C. min 1 in the range of room temperature (RT) to 800° C. Weight loss due to complete degradation of organic content (grafted PS layer) in the range of 200° C. to 600° C. were determined for sample particles after each cycle of adhesion stability test to determine the adhesion stability of grafted PS content onto glass bead surface. The grafted polymerized styrene samples after recovery, washing and drying showed no loss in weight indicating highly stable adhesion of PS onto the glass bead surface. TGA data for the above-mentioned sample after complete removal of unbound PS by Soxhlet extraction and after each cycle of adhesion stability test has been shown in FIG. 5.

It is evident from the TGA analysis that polymerized styrene grafted onto the glass bead surface started degrading after 250° C. and complete combustion happened by 500° C. This further confirms the presence of polymerized styrene on the glass bead surface. Considering the possibility of inhomogeneity of grafted polystyrene around glass beads, average of the TGA data for six different samples was taken to monitor the quantity of polymerized styrene grafted onto the glass bead surface. The quantity of grafted polystyrene for all the samples analyses has been provided in Table 1. Thus, the TGA results also confirmed that the quantity of polymerized styrene grafted onto glass bead remained unchanged to a great extent, even after recovery, washing and drying of the samples from each cycle of adhesion stability test. This indicates excellent adhesion stability of the polymerized styrene grafted onto glass bead surface in the mixture of phenol and acetone (96:4, w/w) at 70° C. during and after multiple cycles.

TABLE 1
Quantity of polymerized styrene grafted onto the glass bead surface

| Sample* | Grafted PS content (wt. %) |
|---|---|
| As prepared | 7.5 ± 0.6 |
| After 1st cycle of stability test | 6.7 ± 0.8 |
| After 2nd cycle of stability test | 7.1 ± 0.8 |
| After 3rd cycle of stability test | 6.6 ± 0.4 |

EDS mapping of the SEM image also investigated the coverage of glass beads by the grafted polystyrene. EDS or EDAX (Energy-dispersive X-ray spectroscopy) analysis was performed on the gold coated samples using an Oxford make “X-Max-50 SDD” EDS system. The cross sectional image of the grafted PS around the glass bead particles was determined by EDS mapping using the same system. Polymerized styrene grafted glass bead particles were embedded in ‘Epo-thin’ epoxy using a “SimpliMet XPS1” automatic compression mounting system, 150° C. and 300 bar pressure was applied. Samples were then precisely polished to the middle of the glass beads using an “EcoMet 300” automated grinder polisher at room temperature. The embedded sample block was then polished using a silica paper (1000 grit) gently pressing against the cloth. Water was used to reduce the abrasion due to polishing. Both the sample block were then gold coated of about 2.5 nm in thickness and characterized using the said SEM and EDS system. ‘Image J’ was used for image processing.

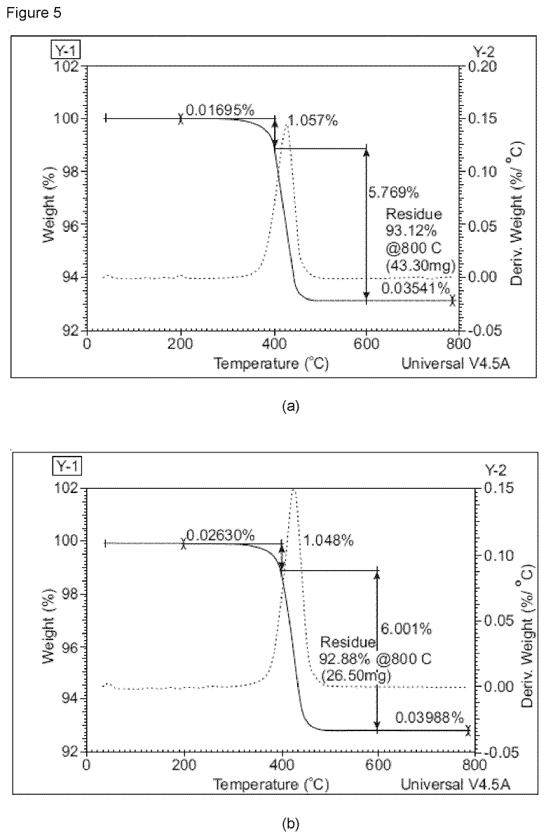

EDS mapping of the cross-sectional image for the polymerized styrene grafted onto the glass bead sample has been shown in FIG. 6. In this figure, the dark color in the center represents glass bead core and the white outside border around the center represents grafted polystyrene moiety. It was apparent from the figure that around the spherical glass bead a thin layer of polystyrene has been coated covering almost all the glass surface. The thickness of the coating was largely non-uniform.

### Catalyst Example 2

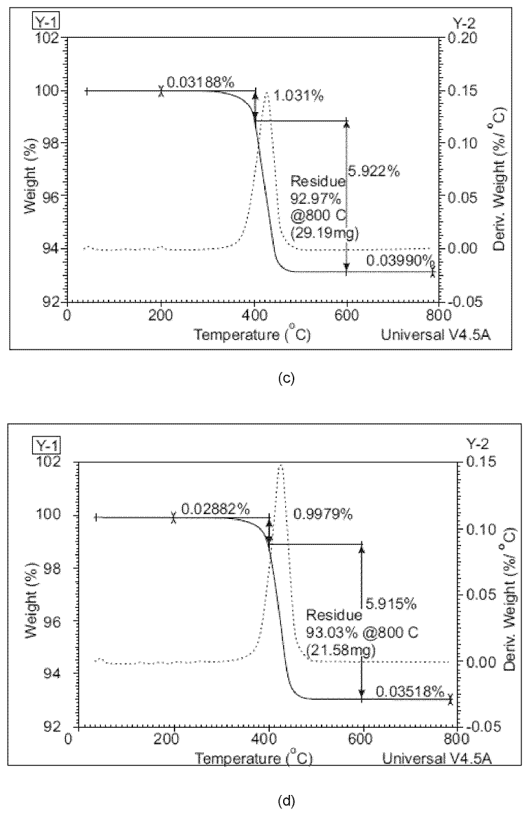

In this example, the samples as obtained at the end of Step 4 (sulfonated polymerized styrene grafted core-shell particles) were analyzed to confirm sulfonation. SEM-EDAX analysis of the samples before and after sulfonation is shown below in FIG. 7. In the figure, the two lines denote samples of polymerized styrene grafted onto the glass bead sample before sulfonation and after sulfonation, as denoted in the spectrum.

TABLE 2
Elemental analysis of polymerized styrene
grafted core-shell particles:

| Element | Before sulfonation (Wt. %) | After sulfonation (Wt. %) |
|---|---|---|
| C | 95.6 | 93.8 |
| O | 4.4 | 5.3 |
| S | 0.0 | 0.88 |

It was observed that after sulfonation reaction, the peak (at 2.3 unit) intensity corresponding to the signal of sulfur increased significantly compared to the sample before sulfonation. This confirmed the presence of sulfur indicating sulfonation of PS moiety. Sulfonation was further confirmed by elemental analysis (Table 2).

### Catalyst Example 3

In this example, the catalyst performance of the core-shell ion exchange resin catalyst (as prepared following Steps 1-4) was evaluated. BPA conversion reaction from the mixture of phenol and acetone (96:4, w/w) using the manufactured core-shell type ion exchange resin catalyst particles (as obtained at the end of Step 4) was carried out at 70° C. for multiple times to evaluate the repeatability and reproducibility of the catalyst performance. Reactions were continued for 8 hours and samples were taken in an interval of one hour to understand the reaction kinetics. Commercially available LEWATIT K-1131® (IER) beads were used as a comparative example. The LEWATIT K-1131@ IER catalyst was pre-dried at 95° C. under vacuum for 8 hours and stored inside phenol for 12 hours to pre-swell prior to use.

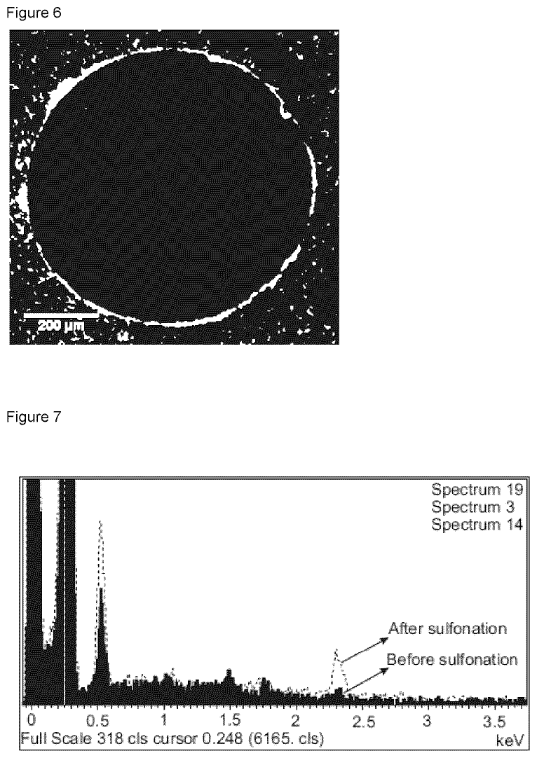

In a typical reaction, 20 gm of reaction mixture contains 96 wt. % phenol and 4 wt. % acetone with the loading of 2 wt. % catalyst. In case of manufactured core-shell catalyst, the quantity of the shell was only considered as catalyst and same amount of IER (LEWATIT K-1131@) was used as comparative example. A comparison of catalytic performance of the manufactured core-shell catalyst with commercially available benchmark catalyst is shown in FIG. 8. It was observed that the catalytic activity of the newly developed catalyst demonstrated excellent catalytic performance in terms of consistent yield of p,p-BPA during multiple repetitive cycles. It also seemed to show a also higher conversion rate as shown in p,p-BPA yield during early hours and high adhesion stability of the covalently grafted catalytic layer onto the glass bead surface.

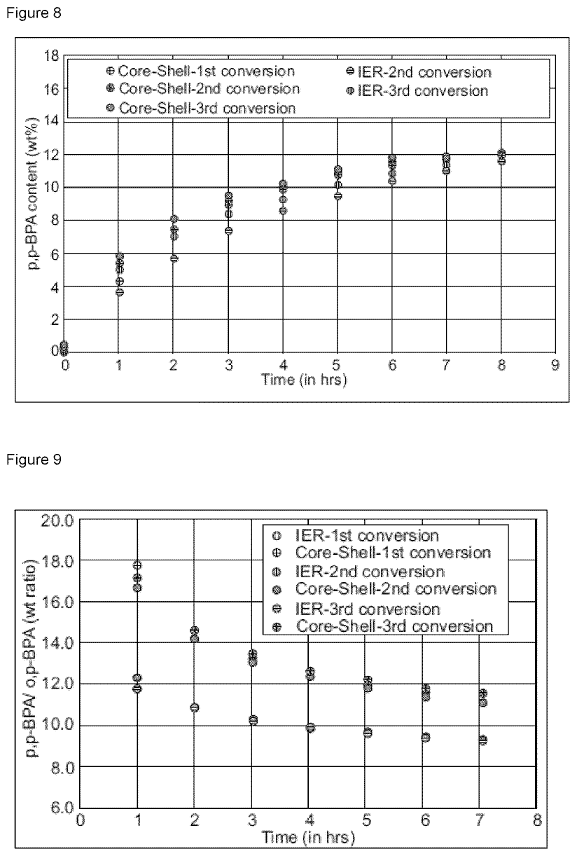

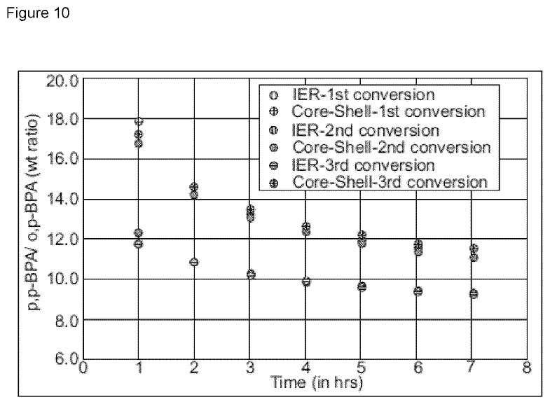

Selectivity towards p,p-BPA formation with respect to o,p-BPA is shown in FIG. 9. Advantage of a thin shell of catalytic layer around the solid core glass bead is again reflected in higher selectivity of p,p-BPA with respect to o,p-BPA and total mixture. The cycles were repeated thrice to confirm the repeatability of the catalytic performance. The performance of the comparative example IER catalyst vis-à-vis the core shell catalyst was compared for the BPA conversion up to sixth cycle, which is shown in FIG. 10. It was observed that the core shell catalyst according to the present invention showed the better performance in terms of yield and selectivity compared to IER based catalyst even up to sixth cycle. This observation demonstrated high integrity of the catalytic layer supported on glass beads.

While the experiments with the catalyst were not performed in an upflow reactor specifically the present inventors believe there is no reason to assume that the the core-shell catalysts disclosed herein perform better from a purely catalytical perspective depending on the mode of operation of the reactor being used.

1. Method for the manufacture of a core-shell catalyst comprising the steps of
a) providing core particles,
b) functionalizing at least part of the surface of said core particles with a functionalizing agent thereby forming functionalized core particles,
c) graft polymerizing one or more aromatic vinyl compounds onto the functionalized core particles thereby forming core-shell particles wherein the core is comprised of said core particles and the shell is comprised of graft polymerized aromatic vinyl compounds, and
d) activating the shell, wherein prior to step b) the core particles are hydroxylated.

2. The method of claim 1, wherein the core particles have an average particle diameter of 200-2000 μm, as determined with microscopy in accordance with the method described in the description.

3. The method of claim 1, wherein the functionalizing agent comprises at least one functional group for reacting with the core particle and at least one functional group for reacting with said one or more aromatic vinyl compounds.

4. The method of claim 1, wherein the functionalizing agent is a silane represented by the following general formula (1), 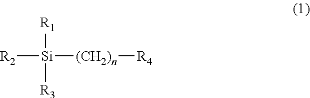
wherein,
R1, R2 and R3 may be the same or different, and are independently selected from the group consisting of hydrogen, halogen, alkyl, haloalkyl, alkylene, alkynyl, aromatic groups, substituted aromatic groups, heteroaromatic groups, ester, ether, allyl, alkoxy, alkynoxy, aryloxy, acyloxy, acrylate, methacrylate, isocyanate, urethane, carbamate, epoxy, carboxylic acid, carboxylic acid anhydride, carboxylate, hydroxy, thiol, amine, aminoalkyl, arylammo and amide provided that at least one of R1, R2 and R3 is an acrylate, allyl, alkoxy, alkynoxy, aryloxy, acyloxy, methacrylate, isocyanate, urethane, carbamate, epoxy, carboxylic acid, carboxylic acid anhydride, carboxylate, hydroxy, thiol, amine, aminoalkyl, arylammo and amide,
R4 is a functional group for reacting with the said aromatic vinyl compounds, and
n is between 0-20.

5. The method of claim 1, wherein the aromatic vinyl compound comprises styrene and optionally a substituted styrene.

6. The method of claim 1, wherein the activating step comprises sulfonating at least part of the graft polymerized aromatic vinyl compound using a sulfonating agent.

7. The method of claim 1, wherein the shell has a thickness from 10-500 μm.

8. Method for carrying out a catalysed chemical reaction using one or more liquid reactants in an upflow reactor comprising:
feeding at least a portion of said reactants to a bottom section of the reactor positioned below a flow distributor plate,
passing said portion through the flow distributor plate,
passing said portion through a layer of inert particles positioned above said flow distributor plate,
passing said portion through a catalyst layer comprising a particulate catalyst, said catalyst layer being positioned above and in contact with said layer of inert particles, wherein the reactants react to form a product stream,
collecting said product stream via collecting means positioned above said catalyst layer,
wherein,
the upflow reactor is operated at a weight hourly space velocity of at least 1.5, and
the inert particles have a density of at least 2000 kg/m3 and an average particle size of from 500 to 5000 μm, and
the particulate catalyst comprises or consists of a core-shell catalyst obtained or obtainable by the method of claim 1,
the height of the layer of inert particles is at least 40 times the average particle size of the inert particles.

9. The method of claim 8, wherein the catalysed chemical reaction comprises the reaction between phenol and ketone.

10. The method of claim 8, wherein the inert particles comprise, essentially consist or consist of particles selected from sand particles, glass particles, ceramic particles, diatomaceous earth particles, inert metal particles and combinations of at least two hereof.

11. The method of claim 8, wherein the distributor plate is a slotted plate with a plurality of openings having a size smaller than the average particle size of the inert particles.

12. The method of claim 1, wherein the height of the layer of inert particles is at least 2.0 cm.

13. A reactor assembly for carrying out a catalysed chemical reaction using one or more liquid reactants in upflow comprising:
feeding means for feeding at least a portion of said reactants to a bottom section of the reactor, said feeding means being positioned below a flow distributor plate,
layer of inert particles positioned above said flow distributor plate,
a catalyst layer comprising a particulate catalyst positioned above and in contact with said layer of inert particles,
collecting means positioned above said catalyst layer for collecting a product stream from the reactor,
wherein,
the inert particles have a density of at least 2000 kg/m3 and an average particle size of from 500 to 5000 μm, and
the particulate catalyst comprises or consists of a core-shell catalyst obtained or obtainable by the method of claim 1,
the height of the layer of inert particles is at least 40 times the average particle size of the inert particles.
